(19) United States
(12) Patent Application Publication
(10) Pub. No.: US 2013/0130373 A1
(43) Pub. Date: May 23, 2013

(54) Kit Comprising Serum Replacement and Labile Factors

(71) Applicant: Essential Pharmaceuticals, LLC, Newtown, PA (US)

(72) Inventors: Adam Elhofy, Chicago, IL (US); Allan Weber, Robbinsville, NJ (US)

(73) Assignee: ESSENTIAL PHARMACEUTICALS, LLC, Newtown, PA (US)

(21) Appl. No.: 13/673,739

(22) Filed: Nov. 9, 2012

Related U.S. Application Data
(60) Provisional application No. 61/558,740, filed on Nov. 11, 2011.

(51) Int. Cl. C12N 5/00

(52) U.S. Cl. CPC C12N 5/0031
USPC 435/346; 435/405; 435/406; 435/358; 435/365; 435/369; 435/367; 435/357

(57) ABSTRACT

The present disclosure relates, in general to a kit comprising a serum replacement and one or more labile factors, such as growth factors, packaged separately in the kit. It is contemplated that the kit provides advantages to improve cell growth in culture compared to cells cultured not using the kit described herein.

The present application claims the priority benefit of U.S. Provisional Patent Application No. 61/558,740, filed Nov. 11, 2011, incorporated by reference herein in its entirety.

### FIELD OF THE INVENTION

The present disclosure, relates, in general, to a kit for culturing cells comprising a serum replacement and one or more labile factors, such as growth factors, cytokines, or hormones, wherein the serum replacement and labile factors are packaged separately in the kit. The kit provides for extended shelf life of the components and improved efficacy and consistency of cell growth in culture.

### BACKGROUND OF THE INVENTION

Culture of cells, e.g., mammalian cells or insect cells, for in vitro experiments or ex vivo culture for administration to a human or animal is an important tool for the study and treatment of human diseases. Cell culture is widely used for the production of various biologically active products, such as viral vaccines, monoclonal antibodies, polypeptide growth factors, hormones, enzymes and tumor specific antigens. However, many of the media or methods used to culture the cells comprise components that can have negative effects on cell growth and/or maintenance of an undifferentiated cell culture. For example, mammalian or insect cell culture media is often supplemented with blood-derived serum, such as fetal calf serum (FCS) or fetal bovine serum (FBS,) in order to provide growth factors, carrier proteins, attachment and spreading factors, nutrients and trace elements that promote proliferation and growth of cells in culture. However, the factors found in FCS or FBS, such as transforming growth factor (TGF) beta or retinoic acid, can promote differentiation of certain cell types (Ke et al., Am J Pathol. 137:833-43, 1990) or initiate unintended downstream signaling in the cells that promotes unwanted cellular activity in culture (Veldhoen et al., Nat Immunol. 7(11):1151-6, 2006).

Additionally, the uncharacterized nature of the serum composition and lot-to-lot variation of the serum make use of a serum replacement and culture in serum-free media desirable (Pei et al., Arch Androl. 49(5):331-42, 2003). Moreover, for cells, recombinant proteins or vaccines for therapeutic use that have been grown in cell culture, the addition of animal-derived components is undesirable due to potential virus contamination and/or to the potential immunogenic effect of the animal proteins when administered to humans.

Serum replacements have been developed in attempts to minimize the effects of FCS on cell culture, as well as minimize the amount of animal protein used for culture of human cells. Serum replacement, such as KNOCKOUT™ serum replacement (Invitrogen, Carlsbad, Calif.), is termed a chemically defined culture medium, lacking serum and containing essential nutrients and other proteins for cell growth. KNOCKOUT SR™ contains protein factors, all of which have a short half life included in the commercial formulation. KNOCKOUT SR™ cannot be used as a replacement for FBS in the plating of feeder cells due to the lack of attachment factors, which results in inadequate cell attachment in this formulation. PC-1™ serum free media (Lonza, Walkersville, Md.) is a low-protein, serum-free medium formulated in a specially modified DMEM/F12 media base and contains a complete HEPES buffering system with known amounts of insulin, transferrin, fatty acids and proprietary proteins. The transferrin in PC-1 media exhibits a half life of 2-4 weeks in solution.

Cellgro COMPLETE™ (Cellgro, Manassas, Va.) is a serum-free, low-protein formulation based on a mix of DMEM/F12, RPMI 1640 and McCoy's 5A. Cellgro COMPLETE™ does not contain insulin, transferrin, cholesterol, growth or attachment factors. Cellgro COMPLETE™ comprises a mixture of trace elements and high molecular weight carbohydrates, extra vitamins, a non-animal protein source, and bovine serum albumin (1 g/L). Cellgro FREE™ (Cellgro, Manassas, Va.) is a serum-free and protein-free growth medium that does not contain any hormones or growth factors.

Serum-free medias are also described in International Patent Publication Nos. WO2009023194, WO2008137641, WO2006017370, WO2001011011, WO2007071389, WO2007016366, WO2006045064, WO2003064598, WO2001011011, US Patent Publication Nos. US20050037492, US20080113433, US20080299540, U.S. Pat. Nos. 5,324,666, 6,162,643, 6,103,529, 6,048,728, 7,709,229 and European Patent Application No. EP2243827.

U.S. Pat. No. 7,220,538 describes a cell culture media comprising lipophilic nanoparticles and base nutritive media.

### SUMMARY

The present disclosure provides a kit comprising reagents for culture of cells in vitro. The kit provides serum replacement and labile factors, packaged in separate containers, which when used for cell culture allows for improved growth and consistency of cells grown using the reagents provided in the kit described herein.

In various aspects, the disclosure provides a kit for improved culture of cells in vitro comprising a first container comprising a serum replacement and one or more separate containers comprising at least one labile factor, such as a growth factor, and instructions for use.

In various embodiments, the serum replacement comprises, i) liposomes and ii) base nutritive media. In a related embodiment, the liposome is a nanoparticle.

In various embodiments, the liposomes comprise lipids, fatty acids, sterols and/or free fatty acids. In various embodiments, the nanoparticle has a mean diameter ranging from about 50 to 500 nm, from about 100 nm to about 300 nm or from about 100 to 200 nm.

In various embodiments, the serum replacement is added to a basic media prior to cell culture. Standard basic media are known in the art and commercially available. Examples of such media include, but are not limited to, Dulbecco's Modified Eagle's Medium (DMEM), DMEM F12, Iscove's Modified Dulbecco's Medium, Ham's Nutrient mixture F-10, Roswell Park Memorial Institute Medium (RPMI), MCDB 131, Click's medium, McCoy's 5A Medium, Medium 199, William's Medium E, and insect media such as Grace's medium and TNM-FH.

Any of these media are optionally supplemented with salts, amino acids, vitamins, buffers, nucleotides, antibiotics, trace elements, and glucose or an equivalent energy source. Other optional supplements may also be included at appropriate concentrations that would be known to those skilled in the art. Media supplements are well-known in the art and commercially available, and are described in greater detail in the Detailed Description.

In various embodiments, it is further contemplated that the serum replacement itself comprises the elements of a base media and supplements as described above, e.g., salts, amino acids, vitamins, buffers, nucleotides, antibiotics, trace elements, and glucose or an equivalent energy source, such that the serum replacement is provided as a serum-free complete media.

In various embodiments, the labile factor is in frozen, liquid or lyophilized form.

In various embodiments, the labile factor is a growth factor, cytokine, a chemokine, a hormone (steroid hormone or peptide hormone), an iron transporter, a peptide factor or a steroid.

In various embodiments, the hormone is selected from the group consisting of insulin, somatastatin, growth hormone, hydrocortisone, dexamethasone 3,3′,5-Triiodo-L-thyronine, and L-Thyroxine.

In various embodiments, the labile factor is a growth factor selected from the group consisting of insulin growth factor (IGF), epidermal growth factor (EGF), fibroblast growth factor (FGF), somatostatin, and triiodo-L-thyronine. Additional growth factors contemplated for use in the kit are known in the art and described further in the Detailed Description.

In various embodiments, the labile factor is a human labile factor. In various embodiments, the labile factor is a rodent (e.g., mouse, rat) labile factor.

In various embodiments, the labile factor is packaged such that when it is added to the serum replacement a final concentration of the labile factor is in the range from about 0.05 to 250 ng/ml, from about 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml. It is further contemplated that the labile factor is in a final concentration of about 0.05, 0.1, 0.5, 1, 2, 3, 4, 5, 6, 7, 8, 9, or 10 ng/ml.

In various embodiments, the growth factor is packaged such that when it is added to the serum replacement a final concentration of the growth factor is in the range from about 0.05 to 250 ng/ml, from about 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml. It is further contemplated that the growth factor or cytokine is in a final concentration of about 0.05, 0.1, 0.5, 1, 2, 3, 4, 5, 6, 7, 8, 9, or 10 ng/ml. In various embodiments, the growth factor is a human growth factor. In various embodiments, the growth factor is a rodent (e.g., mouse, rat) growth factor.

In various embodiments, the serum replacement further comprises an iron source or an iron transporter. In various embodiments, the iron source or iron transporter is selected from the group consisting of transferrin, lactoferrin, ferrous sulphate, ferrous citrate, ferric citrate, ferric ammonium citrate, ferric ammonium oxalate, ferric ammonium fumarate, ferric ammonium malate and ferric ammonium succinate.

In various embodiments, the serum replacement further comprises a copper source or copper transporter (e.g., GHK-Cu). Exemplary copper sources include, but are not limited to, copper chloride and copper sulfate.

In various embodiments, it is contemplated that the serum replacement and one or more labile factors is not intended to cause differentiation of the cells in culture. In various embodiments, the serum replacement media and one or more labile factors do not cause differentiation of the cells in culture.

In various embodiments, the kit further comprises a container comprising an agent for promoting cell adhesion. In various embodiments, the agent that promotes cell adhesion is selected from the group consisting of collagen, fibronectin, vitronectin, synthetic microcarriers and wrapped carbon tubes.

In various embodiments, the iron source or iron transporter, copper source or cell adhesion agent is packaged such that when it is added to the serum replacement a final concentration of the iron transporter, copper source or cell adhesion agent is in the range from about 0.05 to 250 ng/ml, from about 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml. It is further contemplated that the iron transporter, copper source or cell adhesion agent is in a final concentration of about 0.05, 0.1, 0.5, 1, 2, 3, 4, 5, 6, 7, 8, 9, or 10 ng/ml.

In various embodiments, the labile factor supplement is formulated as a cocktail comprising two, three, four, five or more of IGF, EGF, FGF, transferrin, somatostatin, and triiodo-L-thyronine. In various embodiments, the FGF is basic FGF (bFGF, FGF-2) or acidic FGF (aFGF, FGF-1).

In various embodiments, the growth factors in the cocktail are packaged such that when added to the serum replacement a final concentration of IGF is from 0.5 to 3 ng/ml, a final concentration of EGF is from 1-10 ng/ml, a final concentration of FGF is from 3-10 ng/ml, a final concentration of transferrin is from 3-10 ng/ml, a final concentration of somatostatin and triiodo-L-thyronine is from 5-15 ng/ml. In various embodiments, the IGF is at a final concentration of 1 ng/ml. In various embodiments, the EGF and FGF are at a final concentration of 5 ng/ml. In various embodiments the transferrin is at a final concentration of 5 ng/ml. In various embodiments, the somatastatin and triiodo-L-thyronine are at a final concentration of 10 ng/ml.

In various embodiments, the kit comprises vitronectin packaged such that when added to the serum replacement the final concentration of vitronectin is at a concentration from 100-500 ng/ml. In various embodiments, the vitronectin is at a final concentration of 250 ng/ml.

In various embodiments, the serum replacement is animal-component free.

In various embodiments, the separately packaged labile factor, such as a growth factor, has a longer half-life when introduced into the serum replacement than the same labile factor when pre-packaged in the serum replacement or a basic media.

In various embodiments, packaging the one or more labile factors separately from the serum replacement improves the growth and consistency of the cell in cell culture compared to cell culture with a media pre-packaged to contain the labile factor. For example, it is contemplated that the appearance of the cells in culture is consistent and the cells expand at a regular rate compared to growth of cells in another media prepackaged to contain the labile factor(s).

In various embodiments, the cells are selected from the group consisting of mammalian cells and insect cells. In various embodiments, the cell is isolated from a mammalian subject. In various embodiments, the cell is a primary culture of a cell line. In various embodiments, the cell is selected from the group consisting of pluripotent stem cells, embryonic stem cells, bone marrow stromal cells, hematopoietic progenitor cells, lymphoid stem cells, myeloid stem cells, T cells, B cells, macrophages, hepatic cells, pancreatic cells, and cell lines.

Mammalian cell lines contemplated include, but are not limited to, CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, a T cell line (e.g., Jurkat), a B cell line (e.g., BJAB, EW36, CA46, ST486 and MC116, Raji, Namalva and Daudi), 3T3, RIN, A549, PC12, K562, PER.C6®, SP2/0, NS-0, U20S, HT1080, hybridomas, cancer cell lines, and other cell lines well-known in the art. Insect cell lines contemplated include, but are not limited to, Sf9, Sf21, HIGH FIVE™, EXPRESSF+®, S2, TnS, TN-368, BmN, Schneider 2, D2, C6/36 and KC cells.

In various embodiments, the serum replacement and the one or more labile factor are combined within 1, 2, 3, 4, 5, 6 or 7 days of use in the cell culture.

In one embodiment, the serum replacement is packaged in a volume of 10 ml, 50 ml, 100 ml, 500 ml or 1L. In a related embodiment, the serum replacement is packaged in a 1×, 5×, 10× or 20× solution.

In various embodiments, the kit further comprises selection or induction factors, including an antibacterial, anti-fungal or anti-microbial agent. Exemplary agents contemplated include, but are not limited to, gentamicin, ampicillin, amphotericin B, penicillin, streptomycin, hygromycin B, kanamycin, neomycin, methotrexate, isopropyl β-D-1-thiogalactopyranoside (IPTG), and other selection or induction factors known in the art, or combinations thereof.

In various embodiments, the container is selected from the group consisting of a tube, vial, ampoule, and bottle. It is contemplated that the container is made from material well-known in the art, including, but not limited to, glass, polypropylene, polystyrene, and other plastics.

In various embodiments, the container is coated to prevent loss of protein activity. Coating includes additives to the container that prevent the growth factor or other protein in a container from adhering to the container wall. Additives include, but are not limited to, non-animal derived carrier proteins, surfactants, amino acids, and sugars. It is contemplated that additives are adapted for the lyophilized forms or the aqueous forms of growth factor.

In various embodiments, the kit further comprises cells packaged in a separate container.

In another aspect, the disclosure contemplates use of a kit as described herein for culture of cells in vitro.

It is understood that each feature or embodiment, or combination, described herein is a non-limiting, illustrative example of any of the aspects of the invention and, as such, is meant to be combinable with any other feature or embodiment, or combination, described herein. Each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE DRAWINGS

FIG. 1 illustrates that culture of cells with media containing serum replacement (SR) in which growth factors were co-manufactured with the serum replacement (combined a significant period of time prior to cell culture) (Medium+10% Co-manufactured SR) does not promote cell proliferation even after stimulation in vitro (FIG. 1A), whereas culture with serum replacement to which growth factors were added just prior to cell culture (Media+10% SR+Growth Factors) results in cell proliferation (FIG. 1B). Proliferation expressed as increase in optical density (OD) at 450 nm.

### DETAILED DESCRIPTION

The present disclosure provides a kit for culture of cells in vitro, comprising a serum replacement media and a labile factor, wherein the serum replacement and the labile factor are packaged separately in the kit. The kit provides for improved cell culture conditions compared to other serum free medias or serum replacements comprising labile factors by packaging the labile factors, such as growth factors, cytokines, hormones and the like, separately from the serum replacement or media composition. The present kit provides advantages over other serum replacements or medias in that separate packaging of the labile factor provides for improved half-life of the labile factor and a more efficient and consistent cell growth in culture. Not to be bound by theory, it is believed that the inclusion of a labile factor, such as growth factors, cytokines, or hormones, in the media when shipped or addition of the labile factor too long prior to cell culture reduces the longevity and potency of the factor when used for cell culture, resulting in sub-optimal growth or survival of the cells in vitro. The present kit overcomes this problem and provides advantages heretofore undisclosed in the art.

### Definitions

As used herein “serum replacement” or “serum replacement media” refers to a composition that can be used in conjunction with a basal media or as a complete media in order to promote cell growth and survival in culture. In various embodiments, serum replacement is used in basal or complete media as a replacement for any serum that is characteristically added to media for culture of cells in vitro. It is contemplated that the serum replacement comprises proteins and other factors for growth and survival of cells in culture. In various embodiments, the serum replacement is added to a basal media prior to use in cell culture. It is further contemplated that, in various embodiments, a serum replacement may comprise a base media and base nutrients such as salts, amino acids, vitamins, trace elements, and the like, such that the serum replacement is useful as a serum-free complete media for cell culture.

As used herein a “basal media”, “base media”, “base medium” or “base nutritive media” refers to a basal salt nutrient or an aqueous solution of salts and other elements that provide cells with water and certain bulk inorganic ions essential for normal cell metabolism and maintains intra- and extra-cellular osmotic balance. In various embodiments, a base media comprises at least one carbohydrate as an energy source, and/or a buffering system to maintain the medium within the physiological pH range. Examples of commercially available basal media include, but are not limited to, Dulbecco's Modified Eagle's Medium (DMEM), Minimal Essential Medium (MEM), Basal Medium Eagle (BME), RPM1 1640, Ham's F-10, Ham's F-12, a-Minimal Essential Medium (aMEM), Glasgow's Minimal Essential Medium (G-MEM), Iscove's Modified Dulbecco's Medium, or a general purpose media modified for use with pluripotent cells, such as X-VIVO (Lonza) or a hematopoeitic base media. A base media can be supplemented with nutrients as described in greater detail in the Detailed Description. A “complete media” is a cell culture medium with growth supplements already added to basal medium.

As used herein, “labile factor” refers to a substance that functions in a specific biochemical reaction or bodily process and that can undergo a chemical change, for example, such that the factor can be degraded over time. Exemplary labile factors include, but are not limited to, growth factors, cytokines, chemokines, hormones (steroid and peptide hormones), iron transporters, peptide factors and steroids.

As used herein, “growth factor” refers to an agent that promotes growth, proliferation or differentiation of cells. Growth factors contemplated include, but are not limited to, such agents as cytokines, chemokines, or peptide growth factors. Growth factors contemplated for use in the present kit are well-known in the art and described further in the Detailed Description. In various embodiments, the growth factor is a human growth factor. In various embodiments, the growth factor is a rodent (e.g., mouse, rat) growth factor.

In various embodiments, growth factors or labile factors are general or non-specific growth factors that promote growth of most cell types. In one embodiment, the growth factor is selected from the group consisting of insulin growth factor, epidermal growth factor, fibroblast growth factor, somatostatin, triiodo-L-thyronine., interleukin (IL)-2, IL-6 or IL-3. In other embodiments, the growth factor is specific to promote growth of a particular cell type.

In various embodiments, the labile factor is supplied as a single factor or as a mixture comprising two or more labile factors. A mixture of two or more labile factors is referred to herein as a labile factor cocktail. In various embodiments, the labile factor cocktail comprises two or more growth factors.

It is contemplated that when the labile factors are packaged, they are packaged such that when added to the serum replacement the labile factor is at a final concentration appropriate for use in cell culture. It is understood that if the specification refers to a concentration of a labile factor, it is referring to the final concentration of that factor as it is used in the serum replacement or cell culture media.

As used herein, “liposome” refers to a closed structure comprising an outer lipid bi- or multi-layer membrane surrounding an internal aqueous space. Liposomes may be multi-laminar or unilaminar. The liposome is contemplated to range in size from 5 to 10 μm in diameter to nanoparticle size. In certain embodiments, the liposome nanoparticle is from about 50 to 500 nm, from about 100 nm to 300 nm or from about 100 to 200 nm in diameter.

As used herein “improved culture of cells” refers to the increased proliferation of cells, increased growth of cells, decreased cell death, or increased protein production (recombinant or endogenous) of cells when cultured using a kit described herein compared to culture of the cells not using a kit described herein, e.g., using a basal medium comprising serum replacement with growth factors added to the medium upon manufacture, and not compared to culture of cells in medium plus an appropriate amount of serum. Increased proliferation, increased growth and changes in cell death are measured using methods well-known in the art, including growth curve analysis, microscopic evaluation by trypan blue, tritiated thymidine (³H) proliferation assay, MTT assay, resazurin based assays and DNA laddering analysis. Increased protein production of cells is measured using techniques known in the art, including quantitation of total protein or mRNA, or quantitation of levels of a particular protein of interest.

As used herein, the term “do not cause differentiation of the cells” or “not intended to cause differentiation of cells” refers to a state of development of the cell in culture, wherein cells cultured using the kit herein do not take on the characteristics of another cell type or differ substantially in the morphology, profile of protein production or cell surface marker expression as a result of use of the kit herein. For stem cells and progenitor cells, culturing cells such that they do not differentiate is used herein to mean that the cells can proliferate in culture, but the cells remain substantially undifferentiated and express markers of stem or progenitor cells after cell culture. For example, a stem cell or other progenitor cell is “undifferentiated” when a substantial proportion of stem cells and their derivatives in the population display morphological characteristics of pluripotent cells, and are able to develop into multiple cell types. Characteristics of pluripotent stem cells are described in US Patent Publication No. 20050037492 and International Patent Publication No. WO 2001/011011. Alternatively, if the cells are already a fully differentiated cell type or a cell line, cell culture using the kit herein does not cause these cells to differentiate as defined above.

As used herein, “animal-component free” refers to a composition in which the components are not derived from animals. It is contemplated that the components are either produced recombinantly or derived from plants or other sources other than isolated directly from an animal. As used herein, animal-component free allows for recombinant production of labile factors in animal-based cell lines.

As used herein, “container” refers to a receptacle for holding a composition such as serum replacement, growth factor or adhesion agent. It is contemplated that a composition useful in a kit described herein is packaged in a container for transport of the kit. Exemplary containers include, but are not limited to, a vessel, vial, tube, ampoule, bottle, flask, and the like. It is further contemplated that the container is adapted for packaging the serum replacement, growth factor or adhesion agent in lyophilized, liquid or frozen form. It is contemplated that the container is made from material well-known in the art, including, but not limited to, glass, polypropylene, polystyrene, and other plastics.

As used herein, the term “pre-packaged” or “pre-packaged with labile factor” refers to a serum replacement or media that was either co-manufactured with labile factors, such as growth factors, such that the media and growth factors were combined at the time of manufacture, or a serum replacement or media to which labile factors were added a significant period of time prior to use, e.g., 4 months, 5 months, 6 months, or up to 1 year or more prior to use. For example, some commercially sold serum replacement or media is manufactured to contain factors that promote cell growth such that the product, when sold, already contains labile factors, such as growth factors or transferrin, combined in the serum replacement or media, i.e., is pre-packaged with the labile factors.

### Serum Replacement

In various embodiments, the serum replacement comprises, i) liposomes and ii) base nutritive media.

Liposomes may be multi-laminar or unilaminar. The liposome is contemplated to range in size from 5 to 10 μm in diameter to nanoparticle size. In some embodiments, the liposomes are nanoparticles. In certain embodiments, the nanoparticles have a mean diameter ranging from about 50 to 500 nm, from about 100 to about 300 nm, or from about 100 to 200 nm. Liposome size can be measured using methods known in the art, including use of a Zetasizer (Malvern Instruments, United Kingdom), which measures particle size as the average diameter value of the entire particles by the dynamic light scattering method.

In some embodiments, the liposomes comprise lipids, fatty acids, sterols and/or free fatty acids. Methods of making liposomes are known in the art including, liquid hydration or solvent spherule preparation for making multi-laminar vesicles (having series of concentric bi-layer of lipid); and sanitation, French press, solvent injection, detergent removal, reverse phase evaporation, calcium induced fusion, microfluidization or freeze-thaw methods to prepare unilaminar vesicles (having a single layer of lipids).

Liposome preparation is described in U.S. Pat. No. 7,220,538, hereby incorporated by reference, U.S. Pat. No. 6,217,899; US Patent Publication No. 20100021531, Lichtenberg et al., Methods Biochem Anal. 33:337-462, 1988; and G. Gregoriadis: “Liposome Technology Liposome Preparation and Related Techniques,” 2nd edition, Vol. I-III, CRC Press.

In various embodiments, the serum replacement is added to a basic media. Standard basic media are known to in the art and commercially available. Examples of basic media include, but are not limited to, Dulbecco's Modified Eagle's Medium (DMEM), DMEM F12 (1:1), Iscove's Modified Dulbecco's Medium, Ham's Nutrient mixture F-10, Roswell Park Memorial Institute Medium (RPMI), MCDB 131, Click's medium, McCoy's 5A Medium, Medium 199, William's Medium E, and insect media such as Grace's medium and TNM-FH.

Any of these media are optionally supplemented with salts (such as sodium chloride, calcium, magnesium, and phosphate), amino acids, vitamins, buffers (such as HEPES), nucleotides (such as adenosine and thymidine), antibiotics (such as gentamicin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, will be apparent to the ordinarily skilled artisan.

In various embodiments, the serum replacement itself comprises the elements of a base media and supplements as described above, e.g., salts, amino acids, vitamins, buffers, nucleotides, antibiotics, trace elements, and glucose or an equivalent energy source, such that the serum replacement is capable of use as a serum-free complete media.

In various embodiments, the serum replacement comprises an iron source or iron transporter. Exemplary iron sources include, but are not limited to, ferric and ferrous salts such as ferrous sulphate, ferrous citrate, ferric citrate, ferric ammonium compounds, such as ferric ammonium citrate, ferric ammonium oxalate, ferric ammonium fumarate, ferric ammonium malate and ferric ammonium succinate. Exemplary iron transporters include, but are not limited to, transferrin and lactoferrin.

In various embodiment, the serum replacement further comprises a copper source or copper transporter (e.g., GHK-Cu). Exemplary copper sources include, but are not limited to, copper chloride and copper sulfate.

In various embodiments, the iron source or copper source is packaged such that when it is added to the serum replacement a final concentration of the labile factor is in the range from about 0.05 to 250 ng/ml, from about 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml. It is further contemplated that the iron source or copper source is in a final concentration of about 0.05, 0.1, 0.5, 1, 2, 3, 4, 5, 6, 7, 8, 9, or 10 ng/ml.

### Labile Factors

It is contemplated that the labile factors contemplated for use in the kit are effective to promote growth and proliferation of cells in vitro. Labile factors include, but are not limited to, such agents as cytokines, chemokines, hormones (steroid or peptide hormones) iron transporters, peptide factors, steroids or a growth stimulating amine, such as histamine. In various embodiments, the labile factor is a human labile factor. In various embodiments, the growth factor is a rodent (e.g., mouse, rat) labile factor.

In various embodiments, labile factors or growth factors are general or non-specific growth factors that promote growth of most cell types. In various embodiments, the growth factor is specific for a particular cell type, e.g., promotes growth of a family of cell types or a particular type of cell, such as lymphocytes or T cells. In various embodiments, the growth factor is a human growth factor. In various embodiments, the labile factor or growth factor is a rodent (e.g., mouse, rat) growth factor.

Exemplary growth factors contemplated for packaging in the kit include, but are not limited to, bone morphogenic protein (BMP)-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, brain derived neurotrophic factor, ciliary neutrophic factor, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor (FGF) 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor (acidic), fibroblast growth factor (basic), growth related protein, growth related protein α, growth related protein β, growth related protein γ, heparin binding epidermal growth factor, hepatocyte growth factor, insulin-like growth factor I, insulin-like growth factor II, insulin-like growth factor binding protein, keratinocyte growth factor, leukemia inhibitory factor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, pre-B cell growth stimulating factor, stem cell factor, transforming growth factor a, transforming growth factor β, transforming growth factor β1, transforming growth factor 01.2, transforming growth factor 132, transforming growth factor β3, latent transforming growth factor β1, transforming growth factor β binding protein I, transforming growth factor β binding protein II, transforming growth factor β binding protein III, and vascular endothelial growth factor.

Exemplary cytokines for packaging in the kit include, but are not limited to, interleukin (IL) -1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN), IFN-γ, tumor necrosis factor (TNF) 0, TNF1, TNF2, TNF-α, macrophage colony stimulating factor (M-CSF), granulocyte-monocyte colony stimulating factor (GM-CSF), granulocyte colony stimulating factor (G-CSF), megakaryocyte colony stimulating factor (Meg-CSF)-thrombopoietin, stem cell factor, and erythropoietin. Chemokines contemplated for use in the kit include, but are not limited to, IP-10 and Stromal Cell-Derived Factor 1α.

Exemplary hormones contemplated for packaging in the kit include, but are not limited to, steroid hormones and peptide hormones, such as insulin, somatastatin, growth hormone, hydrocortisone, dexamethosone, 3,3′,5-Triiodo-L-thyronine, and L-Thyroxine.

In various embodiments, the labile factor is selected from the group consisting insulin growth factor (IGF), epidermal growth factor (EGF), fibroblast growth factor (FGF), somatostatin, triiodo-L-thyronine, interleukin (IL)-2, IL-6 and IL-3.

It is contemplated that the labile factor is included in a concentration appropriate for the cell type in the cell culture. In various embodiments, the growth factor is packaged such that a final concentration of the growth factor or cytokine when added to the media is in the range of from about 0.05 to 250 ng/ml, from about 0.05 to 100 ng/ml, from about 0.05 to 50 ng/ml, from about 0.05 to 10 ng/ml, from about 0.1 to 5 ng/ml, 0.5 to 2.5 ng/ml, or 1 to 5 ng/ml. It is further contemplated that the growth factor or cytokine is in a final concentration of about 0.05, 0.1, 0.5, 1, 2, 3, 4, 5, 6, 7, 8, 9, or 10 ng/ml.

In various embodiments, the labile factor is formulated as a cocktail comprising two, three, four, five, six or more labile factors described herein. In various embodiments, the labile factor supplement is formulated as a cocktail comprising two, three, four, five or more of IGF, EGF, FGF, transferrin, somatostatin, and triiodo-L-thyronine.

In various embodiments, the growth factors in the cocktail are packaged such that, when added to the serum replacement, a final concentration of IGF is from 0.5 to 3 ng/ml, a final concentration of EGF is from 1-10 ng/ml, a final concentration of FGF is from 3-10 ng/ml, a final concentration of transferrin is from 3-10 ng/ml, a final concentration of somatostatin and triiodo-L-thyronine is from 5-15 ng/ml. In various embodiments, the IGF is at a final concentration of 1 ng/ml. In various embodiments, the EGF and FGF are at a final concentration of 5 ng/ml. In various embodiments the transferrin is at a final concentration of 5 ng/ml. In various embodiments, the somatastatin and triiodo-L-thyronine are at a final concentration of 10 ng/ml.

It is contemplated that the serum replacement media and one or more labile factor are intended to not cause differentiation of the cells in culture. In various embodiments, the serum replacement media and one or more labile factors do not cause differentiation of the cells in culture.

In various embodiments, the kit further comprises a container comprising a factor for promoting cell adhesion. In various embodiments, the factor that promotes cell adhesion is selected from the group consisting of collagen, fibronectin, vitronectin, gelatin, laminin, synthetic microcarriers and wrapped carbon tubes.

In various embodiments, the cell adhesion agent is packaged such that when it is added to the serum replacement a final concentration of the cell adhesion agent is in the range from about 0.05 to 250 ng/ml, from about 5 to 500 ng/ml, from about 50 to 500 ng/ml, from about 100 to 500 ng/ml, from about 0.05 to 100 ng/ml, from about 0.05 to 50 ng/ml, from about 0.05 to 10 ng/ml, from about 0.1 to 5 ng/ml, from about 0.5 to 2.5 ng/ml, or from about 1 to 5 ng/ml. It is further contemplated that the cell adhesion agent is in a final concentration of about 0.05, 0.1, 0.5, 1, 2, 3, 4, 5, 6, 7, 8, 9, or 10 ng/ml.

In various embodiments, the kit comprises vitronectin packaged at a final concentration range from 100-500 ng/ml. In various embodiments, the vitronectin is at a final concentration of 250 ng/ml.

Synthetic microcarriers are known in the art, and include hydrogels, alpha-hydroxy acid family polymers, such as polylactic acid, polyglycolic acid, are polycaprolactone and mixtures thereof. Exemplary microcarriers include, but are not limited to, poly(D,L-lactide-co-glycotide) microcarriers, poly(methyl methacrylate) (PMMA) microspheres, alginate microgels, and gelatin microspheres. Exemplary wrapped carbon tubes, such as carbon nanotubes (CNT), are known in the art and described in U.S. Pat. Nos. 5,753,088, 5,641,466; 5,292,813 and 5,558,903 and US Patent publication No. 20090148417, which describes carbon nanotubes, such as fullerene, carbon buckyball (buckminsterfullerene), carbon nanotube, carbon nanofiber and carbon nanoparticle. Carbon nanotubes are useful as a multilayered shell, a multi-wall nanotube or a single-wall nanotube. In some embodiments, the carbon nanotube is functionalized. Exemplary functional groups linked to CNT include thiol and carboxyl groups. DNA wrapped carbon tubes are described in Lee et al., Angewandte Chemie International Edition 48: 5116-5120, 2009.

In various embodiments, the labile factor is in lyophilized, liquid or frozen form. Methods for preserving labile factors in these different forms is well-known in the art. For example, methods of lyophilizing protein or other material is described in Tang et al., Pharm Res. 21:191-200, (2004) and Chang et al., Pharm Res. 13:243-9 (1996). Lyophilized material can be reconstituted by adding back a volume of pure water or sterile water for injection (WFI) (typically equivalent to the volume removed during lyophilization), or other appropriate buffer [Chen, Drug Development and Industrial Pharmacy, 18:1311-1354 (1992)]. Labile factors for liquid or frozen formulation are prepared in an appropriate buffered solution at a desired concentration that prevents aggregation or precipitation of the growth factor as determined by one of ordinary skill

### Cell Culture

It is contemplated that the kit described herein is useful for culture of cells in vitro, preferably for cells that typically require serum supplements or defined media for adequate growth in vitro. Such cells include eukaryotic cells such as mammalian and insect cells. Mammalian cells contemplated to benefit from use of the kit include, without limitation, hamster, monkey, chimpanzee, dog, cat, bovine, porcine, mouse, rat, rabbit, sheep and human cells. Insect cells include cells derived from Spodoptera frugiperda (caterpillar), Aedes aegypti (mosquito), Aedes albopictus (mosquito), Drosophila melanogaster (fruitfly), and Bombyx mori.

It is contemplated that the cells cultured with the serum replacement are immortalized cells (a cell line) or non-immortalized (primary or secondary) cells, and can be any of a wide variety of cell types that are found in vivo, e.g., fibroblasts, keratinocytes, epithelial cells, ovary cells, endothelial cells, glial cells, neural cells, formed elements of the blood (e.g., lymphocytes, bone marrow cells), chondrocytes and other bone-derived cells, hepatocytes, pancreas cells, and precursors of these somatic cell types.

In various embodiments, the cells contemplated for use with the kit are isolated from a mammalian subject. Cells isolated from a mammalian subject include, but are not limited to, pluripotent stem cells, embryonic stem cells, bone marrow stromal cells, hematopoietic progenitor cells, lymphoid stem cells, myeloid stem cells, lymphocytes, T cells, B cells, macrophages, endothelial cells, glial cells, neural cells, chondrocytes and other bone-derived cells, hepatocytes, pancreas cells, precursors of somatic cell types, and any carcinoma or tumor derived cell.

In various embodiments, the cells are a cell line. Exemplary cell lines include, but are not limited to, Chinese hamster ovary cells, including CHOK1, DXB-11, DG-44, and CHO/-DHFR; monkey kidney CV1, COS-7; human embryonic kidney (HEK) 293; baby hamster kidney cells (BHK); mouse sertoli cells (TM4); African green monkey kidney cells (VERO); human cervical carcinoma cells (HELA); canine kidney cells (MDCK); buffalo rat liver cells (BRL 3A); human lung cells (W138); human hepatoma cells (Hep G2; SK-Hep); mouse mammary tumor (MMT); TRI cells; MRC 5 cells; FS4 cells; a T cell line (Jurkat), a B cell line, mouse 3T3, RIN, A549, PC12, K562, PER.C6®, SP2/0, NS-0, U20S, HT1080, hybridomas, tumor cells, and immortalized primary cells.

Exemplary insect cell lines, include, but not limited to, Sf9, Sf21, HIGH FIVE™ EXPRESSF+®, S2, TnS, TN-368, BmN, Schneider 2, D2, C6/36 and KC cells.

Serum replacement and cell culture conditions contemplated in the present kit may be adapted to any culture substrate suitable for growing cells. Substrates having a suitable surface include tissue culture wells, culture flasks, roller bottles, gas-permeable containers, flat or parallel plate bioreactors or cell factories. Also contemplated are culture conditions in which the cells are attached to microcarriers or particles kept in suspension in stirred tank vessels.

Cell culture methods are described generally in the Culture of Animal Cells: A Manual of Basic Technique, 6^{th} Edition, 2010 (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other reference texts include Creating a High Performance Culture (Aroselli, Hu. Res. Dev. Pr. 1996) and Limits to Growth (D. H. Meadows et al., Universe Publ. 1974). Tissue culture supplies and reagents are well-known to one of skill and commercially available.

It is understood that the cells are placed in culture at densities appropriate for the particular cell line or isolated cell type used with the components of the kit. In certain embodiments the cells are cultured at 1×10³, 5×10³, 1×10⁴, 5×10⁴, 1×10⁵, 5×10⁵, 1×10⁶, or 5×10⁶ cells/ml.

In various embodiments, the serum replacement and the one or more labile factor or cytokine are combined within 1, 2, 3, 4, 5, 6 or 7 days of use in the cell culture.

It is contemplated that packaging the labile factor separately from the serum replacement improves the efficacy of the labile factor in cell culture compared to a media packaged already comprising the labile factor. For example, it is contemplated that the half-life of the labile factor is longer when used as in the present kit compared to media comprising the labile factor. Further, it is contemplated that packaging the one or more labile factors separately from the serum replacement improves the growth of the cells in cell culture compared to cells cultured with media pre-packaged with the labile factor.

In various embodiments, the serum replacement and labile factor compositions are packaged in a container, such as a sealed bottle or vessel or other container disclosed herein, with a label affixed to the container or included in the package that describes use of the compositions for use in vitro, in vivo, or ex vivo. In various aspects, the compositions are packaged in a unit dosage form. The kit optionally includes a device suitable for combining the labile factor with the serum replacement, and alternatively combining the labile factor and serum replacement with a basic media. In various aspects, the kit contains a label that describes use of the labile factor and serum replacement for cell culture.

It is further contemplated that the kit is packaged into suitable packaging material. The term “packaging material” refers to a physical structure housing the components of the kit. The packaging material can maintain the components sterilely, and can be made of material commonly used for such purposes (e.g., paper, corrugated fiber, glass, plastic, foil, ampoules, and other materials known in the art).

Additional aspects and details of the present kit will be apparent from the following examples, which are intended to be illustrative rather than limiting.

### EXAMPLES

### Example 1

Serum Replacement with Fresh Labile Factor Promotes Cell Proliferation In Vitro

In order to test the ability of the serum replacement to promote growth of cells in culture, B cells were isolated from a mouse spleen using standard techniques and stimulated to proliferate using bacterial lipopolysaccharide (LPS) (100 ng/ml).

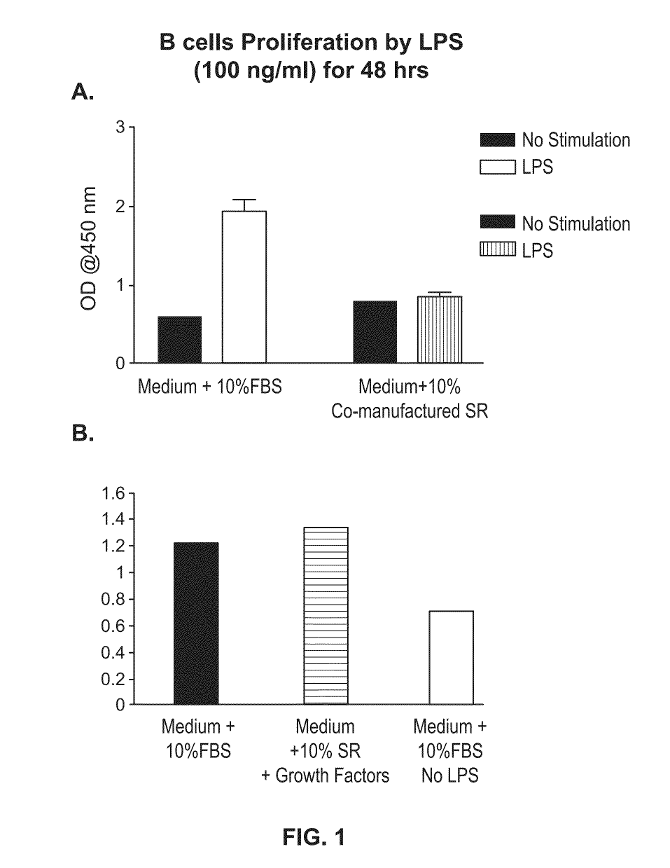

Briefly, single cell suspensions from spleens were isolated from mice by mechanical disruption through mesh stainless steel screens. Red blood cells in the spleen samples were lysed by hypotonic shock in Tris-NH4Cl (pH 7.3) and cells were resuspended in HBSS. Cells were then washed again and cultured in 96-well plates (Corning-Costar, Acton, Mass.) at a density of 5×10⁶ viable cells/ml in DMEM (Life Technologies) [2 mM L-glutamine (Life Technologies, Carlsbad, Calif.), 100 U/ml penicillin (Life Technologies), 100 μg/ml streptomycin (Life Technologies), 0.1 M nonessential amino acids (Life Technologies) and 5×10-5M 2-ME)] containing 10% FBS (HyClone, Logan Utah) or serum replacement, used here as a complete media containing a base media and essential nutrients. The serum replacement used in FIG. 1A was serum replacement pre-packaged or manufactured with labile factor that had been combined over a six months prior to the performance of the experiment. The serum replacement in FIG. 1B was serum replacement in which labile factors (FGF, EFG, and IGF and transferrin) had been added shortly before culture (e.g., within 1 day). Cells were incubated at 37° C. in a humidified atmosphere containing 7.5% CO₂.

FIG. 1A shows that media +10% FBS allowed for significant proliferation of B cells when stimulated by LPS. Culture of B cells in serum replacement containing labile factors packaged together more than 6 months prior to the experiment did not provide sufficient nutrients to allow proliferation of B cells even after LPS stimulation (FIG. 1A). In contrast, cells cultured in serum replacement to which labile factors were added just prior to cell culture proliferated as well as cells cultured in media containing 10% FBS (FIG. 1B).

T cells and macrophages isolated from mice and cultured in fresh 10% serum replacement media as described above also exhibited proliferation or activation in cell culture, respectively. In addition, CHO-K1 and A-549 cell lines adapted to and cultured in serum replacement as above demonstrated good proliferative responses.

These results demonstrate that inclusion of labile factors in the cell culture media when packaging the media can lead to inefficient and impaired growth and survival of cells in culture due to breakdown of the growth factor over time. Addition of labile factors to serum replacement shortly before use of the media in cell culture restores the ability of the serum replacement media to enable healthy growth and proliferation of cultured cells.

### Example 2

### Growth of Cell Lines in Serum Replacement and Labile Factors

Growth in serum-free media can require adaptation of particular cell lines to grow in a serum free environment. In order to determine whether cell lines can be adapted to grow in the serum-replacement comprising freshly added labile factors, viability and growth assays were carried out.

Cell adaptation was carried out over a period of 6-10 weeks. CHO-K1 cells and A549-NFkB SEAP cells were seeded in 6 well plates with 2×10⁴ cells per well and time until population doubling and cell viability after 72 hours were measured. At 24, 48 and 72 hours, one well of the plate was harvested and the total number of cells counted by cytometer and viability of the harvested cells assessed by cell morphology under a microscope. Adapted CHO cells were grown in media plus 10% serum replacement, adapted A549 cells were grown in media plus 10% serum replacement. Control wells were grown in media containing 5% FBS.

CHO cells exhibited 95% viability (control, 96% viability) and cell population doubled by approximately 30 hours (control, approximately 24 hours). A549 cells exhibited cell doubling at approximately 3.5 days compared to approximately 2.5 days for control cells. A549 viability and control viability were approximately 98% after 72 hours.

These results demonstrate that cell lines typically grown in media containing FBS can be adapted to grow in media comprising the present serum replacement.

As noted above, addition of fresh labile factor(s) to culture media can improve proliferation and viability of cultured cells. To determine if addition of fresh labile factors has beneficial effects on cell lines cultured in serum replacement media, labile factors were added to culture media alone or in combination and cell proliferation measured using a Resazurin fluorescence assay following the manufacturer's protocol (Sigma, St. Louis, Mo.). An increase in fluorescence (Resazurin fluorescent units, RFU) is indicative of increased cell proliferation in the sample.

Initial growth factors tested included insulin growth factor (IGF), vascular endothelial growth factor (VEGF), fibroblast growth factor (FGF) and epidermal growth factor (EGF). Culture of CHO cells (5×10⁶ cells/ml) in 10% serum replacement media alone or with growth factors, listed at the final concentration in the cell culture media as follows, resulted in the measured RFU: (1) control, Serum replacement (SR) with no growth factor, approximately 1000 RFU, (2) SR+1 ng/ml IGF, approximately 2000 RFU; (3) SR+VEGF and 1 ng/ml IGF, approximately 2200 RFU at all VEGF concentrations tested; (4) SR+FGF and 1 ng/ml IGF, approximately 2700 RFU at 1.5-3.5 ng/ml FGF and approximately 3000 RFU at 5 ng/ml FGF; (5) SR+EGF, approximately 2100 RFU at all EGF concentrations tested.

Addition of transferrin (final concentration 5 ng/ml) alone to serum replacement improved growth of CHO cells to a small extent, but addition of growth factors (IGF and EGF) in addition to transferrin had an improved effect on growth of cultured cells. Addition of trasnsferrin plus IGF and EGF in the serum replacement resulted in a rate of cell proliferation greater than 50% the rate of cells cultured in FBS, e.g., approximately 15500 RFU for SR+growth factors and transferrin compared to approximately 18000 RFU for FBS control. The proliferation observed for the serum replacement plus added factors is a significantly improved proliferation compared to serum replacement alone or other serum replacements commercially available. See e.g., Lund et al., Cytotherapy 11(2):189-97, 2009, which describes that certain serum replacements are inferior to and less consistent than culture in FBS.

FBS provides certain factors such as adherence factors that allow adherent cells to stick to plates more efficiently, thereby improving cell growth and speeding up the time it takes to reach exponential growth in culture. Adherent factors were added to the serum replacement media and the growth of adherent CHO-K1 cells was assessed. Adapted CHO-K1 cells (5×10⁶ cells/ml) were cultured in control media (10% FBS) or 10% serum replacement media plus vitronectin at 250 ng/ml final concentration and cell adherence and morphology visualized. Cells cultured in the presence of vitronectin showed cell morphology comparable with control cells cultured in FBS, and were adhered by approximately 24 hours after culture. Cells cultured in serum replacement without FBS did not adhere until approximately 96 hours after culture and do not spread well on the surface.

Growth hormones are also present in typical FBS used in culture media (Brunner et al., ALTEX 27: 53-62, 2010). To determine if addition of one or more hormones improved cell growth in media containing serum replacement described herein, a mixture of hormones (somatostatin-10 ng/ml, dexamethasone-20 ng/ml, or 3,3,5-trijodo-L-thyronine-10 ng/ml), or growth factors (EGF-10 ng/ml, IGF-1 ng/ml, and FGF-5 ng/ml) (all concentrations given at final concentration in culture media) were added to the culture media. Control cells were cultured in media plus 5% FBS.

Culture of CHO cells in serum replacement containing IGF, and transferrin (5 ng/ml final) plus hormone mix resulted in proliferation measured at approximately 14000 RFU while control proliferation was approximately 20000 RFU. Removal of dexamethasone from the hormone mix had no effect on CHO cell proliferation. Addition of a growth factor mix (EGF, IGF and FGF) to the culture medium containing hormones reduced proliferation to approximately 9500 RFU. These results demonstrate that addition of hormones to the serum replacement can improve proliferation of cells compared to culture in serum replacement containing only IGF and transferrin.

In order to minimize the number of separate vials in the kit contemplated herein, in one aspect the labile factors are formulated in a cocktail comprising two or more of the labile factors packaged separately from the basal serum replacement media. Prior to the present disclosure there was prevailing thought in the field that combining multiple labile factors in a single formulation at a concentration appropriate for culture of cells was unnecessary, complicated and difficult to carry out under good manufacturing practice (GMP) standards. The ability to combine all the necessary factors are beyond the production capabilities of many manufacturers. The inventors, however, have overcome the difficulties in the field and worked with manufacturers to devise a way to manufacture a cocktail comprising more than one labile factor for use in the kit herein.

Numerous modifications and variations in the invention as set forth in the above illustrative examples are expected to occur to those skilled in the art. Consequently only such limitations as appear in the appended claims should be placed on the invention.

1. A kit for improved culture of cells in vitro comprising a first container comprising a serum replacement and one or more separate containers comprising at least one labile factor, and instructions for use.

2. The kit of claim 1, wherein the serum replacement comprises, i) liposomes and ii) base nutritive media.

3. The kit of claim 2, wherein the liposome is a nanoparticle

4. The kit of claim 3, wherein the nanoparticles have a mean diameter ranging from about 50 nm to about 500 nm.

5. The kit of claim 2, wherein the liposome comprises lipids, fatty acids, sterols and/or free fatty acids.

6. The kit of claim 1, wherein the labile factor is in frozen, liquid or lyophilized form.

7. The kit of claim 1, wherein the kit comprises two, three, four, five, or six or more labile factors.

8. The kit of claim 1, wherein the labile factor is selected from the group consisting of a growth factor, cytokine, a chemokine, a steroid hormone, a peptide hormone, an iron transporter, a peptide factor and a steroid.

9. The kit of claim 1, wherein the labile factor is selected from the group consisting of insulin growth factor, epidermal growth factor, fibroblast growth factor, somatostatin, and triiodo-L-thyronine.

10. The kit of claim 9, wherein the labile factor is packaged such that a final concentration of the growth factor when added to the media is in the range of 0.05 to 250 ng/ml.

11. The kit of claim 1, further comprising an iron source or iron transporter.

12. The kit of claim 11, wherein the iron source or iron transporter is selected from the group consisting of transferrin, lactoferrin, ferrous sulphate, ferrous citrate, ferric citrate, ferric ammonium citrate, ferric ammonium oxalate, ferric ammonium fumarate, ferric ammonium malate and ferric ammonium succinate

13. The kit of claim 1, further comprising a copper source.

14. The kit of claim 1, wherein the serum replacement media and one or more labile factors do not cause differentiation of the cells in culture.

15. The kit of claim 1, further comprising a container comprising an agent for promoting cell adhesion.

16. The kit of claim 15, wherein the agent that promotes cell adhesion is selected from the group consisting of collagen, fibronectin, vitronectin, synthetic microcarriers and wrapped carbon tubes.

17. The kit of claim 1, wherein the labile factor supplement is a cocktail comprising two or more of insulin growth factor (IGF), epidermal growth factor (EGF), fibroblast growth factor (FGF), transferrin, somatostatin, and triiodo-L-thyronine.

18. The kit of claim 17, wherein the final concentration of IGF is from 0.5 to 3 ng/ml, the final concentration of EGF is from 1-10 ng/ml, the final concentration of FGF is from 3-10 ng/ml, the final concentration of transferrin is from 3-10 ng/ml, and the final concentration of somatostatin and triiodo-L-thyronine are from 5-15 ng/ml.

19. The kit of claim 16, wherein the vitronectin is at a final concentration range from 100-500 ng/ml.

20. The kit of claim 1, wherein the serum replacement is animal-component free.

21. The kit of claim 1, wherein the separately packaged labile factor has a longer half-life when introduced into serum replacement than the same labile factor when pre-packaged in serum replacement.

22. The kit of claim 1, wherein packaging the one or more labile factors separate from the serum replacement improves the growth of the cell in cell culture compared to culture with a media pre-packaged with the labile factor.

23. The kit of claim 1, wherein the cell is selected from the group consisting of pluripotent stem cells, embryonic stem cells, bone marrow stromal cells, hematopoietic progenitor cells, lymphoid stem cells, myeloid stem cells, T cells, B cells, macrophages, hepatic cells, pancreas cells, a carcinoma cell and cell lines.

24. The kit of claim 23, wherein the cell line is selected from the group consisting of CHO, CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, a T cell line, a B cell line, 3T3, RIN, A549, PC12, K562, PER.C6, SP2/0, NS-0, U20S, HT1080, a hybridoma and a cancer cell line.

25. The kit of claim 1, wherein the serum replacement and the one or more labile factors are combined within 1, 2, 3, 4, 5, 6 or 7 days of use in the cell culture.

26. The kit of claim 1, wherein the serum replacement is packaged in a volume of 50 ml, 100 ml, 500 ml or 1L.

27. The kit of claim 1, wherein the serum replacement is packaged in a 1×, 5×, 10× or 20× solution.

28. The kit of claim 1, further comprising a container comprising a selection or induction agent.

29. The kit of claim 1, wherein the container is selected from the group consisting of a tube, vial, ampoule, and bottle.

30. The kit of claim 1, wherein the container comprising the labile factor is coated to prevent loss of protein activity.

31. The kit of claim 1, wherein the kit further comprises cells packaged in a separate container.

32. The kit of claim 1, wherein the serum replacement and labile factor are added to a basic media.

33. The kit of claim 1, wherein the serum replacement is a complete media.

34. (canceled)
